(19) United States
(12) Patent Application Publication
(10) Pub. No.: US 2026/0199410 A1
(43) Pub. Date: Jul. 16, 2026

(54) Mixture of non-digestible oligosaccharides

(71) Applicant: N.V. Nutricia, Zoetermeer (NL)

(72) Inventors: Cornelus Johannes Petrus Van Limpt, Utrecht (NL); Sebastian Tims, Utrecht (NL); Amra Buco, Utrecht (NL)

(73) Assignee: N.V. Nutricia, Zoetermeer (NL)

(21) Appl. No.: 19/571,578

(22) Filed: Mar. 19, 2026

Related U.S. Application Data
(63) Continuation of application No. PCT/EP2024/077943, filed on Oct. 4, 2024.

(30) Foreign Application Priority Data
Oct. 4, 2023 (EP) 23201615.4

(51) Int. Cl. A61K 35/745; A23L 33/00; A61K 9/00; A61K 35/00; A61K 47/06; A61K 47/26; A61K 47/42; A61P 3/02

(52) U.S. Cl. CPC A61K 35/745; A23L 33/40; A61K 9/0095; A61K 47/06; A61K 47/26; A61K 47/42; A61P 3/02; A61K 2035/115

(57) ABSTRACT

The invention relates to nutritional compositions comprising a mixture of non-digestible oligosaccharides comprising short-chain fructo-oligosaccharides, long-chain fructo-oligosaccharides and 2′-fucosyllactose that have been shown to provide a beneficial short-chain fatty acids profile and intestinal bifidobacteria population. The nutritional compositions are for infants and young children and are in particular beneficial for infants or young children suffering from food allergy, in particular cow's milk protein allergy.

### FIELD OF THE INVENTION

The invention relates to nutritional compositions comprising non-digestible oligosaccharides for infants and young children, in particular allergic infants or young children,

### BACKGROUND OF THE INVENTION

Human milk is the preferred source of early life nutrition. It has been shown to support healthy infant growth and development and to provide protection against several immunological conditions in early life, including various infectious and allergic diseases.

Although breast milk is the preferred nutrition for infants, it is sometimes not desired or not chosen and in that case infant formulas are commercially available that supply nutrition to the infant. Typically such infant formulas contain intact proteins from cow's milk, vegetable oils and as digestible carbohydrate lactose, as the main digestible carbohydrate in human milk is lactose.

However, such formulas may not be suitable for infants that suffer from cow's milk protein allergy or lactose intolerance. The prevalence of food allergy has been increasing in recent decades, particularly in developed countries. In early life, cow's milk allergy (CMA) is the most dominant food allergy with currently 2-5% of infants suffering from it. CMA in infancy represents an increasing global health and economic burden, which is caused not only by an increased prevalence over the last decades, but also by an increased persistence, severity and complexity of the condition. Besides acute clinical manifestations, which can be severe, CMA in early life can also have long-lasting effects, including delays in growth and development, as well as increased risk for the development of atopic diseases later in life. Hence, strategies to treat or prevent CMA are of major importance.

The standard dietary management of CMA in infants and children is allergen avoidance through elimination of cow's milk from the diet. A variety of formulas have been developed for the elimination of cow's milk protein. Extensively hydrolyzed formulas (eHFs) are recommended for infants with mild CMA, whereas for infants with severe CMA and for infants that either do not tolerate eHFs or in which eHFs fail to resolve CMA symptoms, amino acid-based formulas (AAFs) are recommended. Although lactose is the preferred digestible carbohydrate in formulas, products for severe allergic infants typically do not contain lactose as lactose is derived from cow's milk and all ingredients obtained from cow's milk should be avoided in the manufacture of such products.

Human milk contains human milk oligosaccharides and as a functional mimic, infant formulas may contain non-digestible oligosaccharides such as galacto-oligosaccharides (GOS) with long-chain fructo-oligosaccharides (lcFOS). Such a mixture was found to increase bifidobacteria and improve the metabolic profile, making it more reminiscent to the microbiota and the metabolic profile of the intestine of breastfed infant (Oozeer et al, 2013, Am J Clin Nutr 98: 561S-71S)-. However, GOS is prepared from lactose using beta-galactosidase. Commercial preparations of GOS typically contain a substantial amount of milk derived lactose with traces of whey proteins. Hence GOS is less suitable as a non-digestible oligosaccharide in formulas intended for severe allergic infants. For such infants as alternative, a mixture of short-chain fructo-oligosaccharides (scFOS) and long-chain fructo-oligosaccharides (lcFOS) has been developed, such as present in Neocate Syneo.

WO 2018/067002 discloses a nutritional composition comprising indigestible fiber and a lactic acid producing bacterium, for use in normalizing the intestinal microbiota in amino acid-based formula-fed infants or toddlers.

WO 2015/077233 A1 discloses preventing or mitigating an acute allergic response in a subject by administering a nutritional composition that includes at least one of an acidic HMO, such as 6′-siallyllactose (6′-SL) or a neutral HMO, such as 2′-fucosyllactose (2′-FL), but does not include an N-acetyl-lactosamine.

WO 2021/074375 A1 discloses an extensively hydrolysed infant formula (eHF) and amino acid-based infant formula (AAF), comprising the human milk oligosaccharides (HMOs) 2′-fucosyllactose (2′-FL) and/or lacto-N-neotetraose (LNnT), and with an amount of protein closer to that of human breast milk, for use in infants with cow's milk protein allergy.

WO 2020/245311 A2 and WO 2020/245313 A1 disclose nutritional compositions for infants or young children comprising 2′-FL and dietary butyrate or 3′-galactosyllactose (3′-GL), preferably both, and in combination with hydrolysed protein and/or free amino acids, a use for prevention or treating prevention or treating of cow's milk protein allergy is described.

WO 2011/0080086 A1 describes nutritional compositions comprising 2′-FL and beta-galacto-oligosaccharides to stimulate the immune system and also a benefit in case of food allergy is described.

### SUMMARY OF THE INVENTION

The inventors found that fermentation, by microbiota from breastfed infants, of a combination of short-chain fructo-oligosaccharides and long-chain fructo-oligosaccharides (scFOS/lcFOS) when compared with a combination of short-chain galacto-oligosaccharides and long-chain fructo-oligosaccharides (scGOS/lcFOS) resulted in lower amounts of total short-chain fatty acids, acetic acid and lactic acid, and higher amounts of propionic acid.

Surprisingly a mixture of scFOS/lcFOS and 2′-fucosyllactose (2′-FL) resulted in a higher amount of total short-chain fatty acids, acetic acid and lactic acid and a lower amount of propionic acid. The metabolic profile was closer to that observed with scGOS/lcFOS fermentation and as can be expected for infants receiving breast milk. This effect was synergistic, as it could not be expected based on the results with 2′-FL or scFOS/lcFOS alone. Additionally the amount of gas formed was lower than expected.

In a subsequent experiment with faecal material from breastfed infants and from toddlers similar results were obtained. The mixture of scFOS/lcFOS/2′-FL performed better than expected and resulted in an age-appropriate metabolic profile that is associated with less risk of atopic disease and also resulted in reduced proteolytic activity and higher levels of bifidobacteria compared to 2′-FL or scFOS/lcFOS alone.

Even better results were observed with a mixture of scFOS/lcFOS/2′-FL that further included Bifidobacterium breve. This composition performed better than scFOS/lcFOS with B. breve or scFOS/lcFOS/2′-FL without B. breve.

These results are indicative that an advantageous gut health can be obtained with a mixture of scFOS/lcFOS/2′-FL, which is further improved by adding B. breve, and this is of particular benefit in infants and young children with an allergic condition, in particular that have cow's milk protein allergy. Also infants and young children suffering from lactose intolerance benefit from nutrition with a mixture of scFOS/lcFOS/2′-FL. The found gut health benefits are manifest by an advantageous intestinal microbiota activity or in other words an advantageous intestinal microbiota metabolic profile. The present finding provides for formulas without milk derived ingredients for severely allergic infants.

### DETAILED DESCRIPTION OF THE INVENTION

The present invention thus concerns a nutritional composition comprising lipids, digestible carbohydrates, a mixture of non-digestible oligosaccharides comprising short-chain fructo-oligosaccharides (scFOS), long-chain fructo-oligosaccharides (lcFOS) and 2′-fucosyllactose (2′-FL), wherein the wt/wt ratio of the sum of scFOS and lcFOS to 2′-FL is 9:0.2 to 9:2, preferably 9:0.4 to 9:1. Preferably the present nutritional composition does not comprise milk-derived ingredients.

The invention also concerns the use of the nutritional composition according to the invention for providing nutrition to an infant or young child.

The invention also concerns a method for providing nutrition to a human subject suffering from food allergy, preferably suffering from cow's milk protein allergy, comprising administering the nutritional composition according to the invention to the human subject. In other words, the invention concerns the present nutritional composition for use in providing nutrition to a human subject suffering from food allergy, preferably suffering from cow's milk protein allergy.

The invention can also be worded as non-digestible oligosaccharides for the manufacture of a nutritional composition for providing nutrition to a human subject suffering from food allergy, preferably suffering from cow's milk protein allergy, wherein the non-digestible oligosaccharides comprise a mixture of short-chain fructo-oligosaccharides (scFOS), long-chain fructo-oligosaccharides (lcFOS) and 2′-fucosyllactose (2′-FL), wherein the wt/wt ratio of the sum of scFOS and lcFOS to 2′-FL is 9:0.2 to 9:2, preferably 9:0.4 to 9:1.

The invention also concerns a method for providing nutrition to a human subject suffering from lactose intolerance, comprising administering the nutritional composition according to the invention to the human subject. In other words, the invention concerns the present nutritional composition for use in providing nutrition to a human subject suffering from lactose intolerance.

The invention can also be worded as non-digestible oligosaccharides for the manufacture of a nutritional composition for providing nutrition to a human subject suffering from lactose intolerance, wherein the non-digestible oligosaccharides comprise a mixture of short-chain fructo-oligosaccharides (scFOS), long-chain fructo-oligosaccharides (lcFOS) and 2′-fucosyllactose (2′-FL), wherein the wt/wt ratio of the sum of scFOS and lcFOS to 2′-FL is 9:0.2 to 9:2, preferably 9:0.4 to 9:1.

The invention also concerns a method for the dietary management of allergy in a human subject, preferably the dietary management of cow's milk protein allergy, comprising administering the nutritional composition according to the invention to the human subject. In other words, the invention concerns the present nutritional composition for use in the dietary management of allergy in a human subject, preferably the dietary management of cow's milk protein allergy.

The invention can also be worded as non-digestible oligosaccharides for the manufacture of a nutritional composition for the dietary management of allergy in a human subject, preferably the dietary management of cow's milk protein allergy, wherein the non-digestible oligosaccharides comprise a mixture of short-chain fructo-oligosaccharides (scFOS), long-chain fructo-oligosaccharides (lcFOS) and 2′-fucosyllactose (2′-FL), wherein the wt/wt ratio of the sum of scFOS and lcFOS to 2′-FL is 9:0.2 to 9:2, preferably 9:0.4 to 9:1.

The invention also concerns a method for promoting intestinal health and/or preventing disorders related to a disbalanced intestinal microbiota in a human subject, comprising administering the nutritional composition according to the invention to the human subject. In other words, the invention concerns the present nutritional composition for use in promoting intestinal health and/or preventing disorders related to a disbalanced intestinal microbiota in a human subject.

The invention can also be worded as non-digestible oligosaccharides for the manufacture of a nutritional composition for promoting intestinal health and/or preventing disorders related to a disbalanced intestinal microbiota in a human subject, wherein the non-digestible oligosaccharides comprise a mixture of short-chain fructo-oligosaccharides (scFOS), long-chain fructo-oligosaccharides (lcFOS) and 2′-fucosyllactose (2′-FL), wherein the wt/wt ratio of the sum of scFOS and lcFOS to 2′-FL is 9:0.2 to 9:2, preferably 9:0.4 to 9:1.

As defined above, the nutritional composition according to the invention comprises a protein component, a lipid component, a digestible carbohydrate component and a non-digestible oligosaccharide component. The protein component, lipid component, digestible carbohydrate component and non-digestible oligosaccharide are described in more detail below.

scFOS/lcFOS/2′-FL Mixture

The nutritional composition contains a mixture of at least three types of non-digestible oligosaccharides, most preferably a mixture of three non-digestible oligosaccharides [NDO]. NDO are not digested in the intestine by the action of digestive enzymes present in the human upper digestive tract, e.g. small intestine and stomach. NDO are fermented by human intestinal microbiota. For example, glucose, fructose, galactose, rhamnose, sucrose, xylose, lactose, maltose, maltodextrins and glucose syrup are considered digestible carbohydrates. Although the sources of NDO may comprise digestible carbohydrates such as glucose, fructose, galactose, rhamnose, xylose, lactose etc., these are not part of the non-digestible oligosaccharides, but included with the digestible carbohydrates instead.

The NDO in the present nutritional composition are composed of at least two types of fructo-oligosaccharides, namely a combination of long-chain fructo-oligosaccharides with short-chain fructo-oligosaccharides.

The nutritional composition comprises short-chain fructo-oligosaccharides (scFOS). In the context of the present invention scFOS has an average degree of polymerisation (DP) of less than 10, preferably scFOS has an average DP in the range of 2-7. In fructo-oligosaccharides fructose units are coupled to each other by beta1,2 linkages. At the terminal end a glucose may or may not be present. scFOS may be inulin hydrolysate products having an average DP within the aforementioned range. Such scFOS products are for instance commercially available as Raftilose P95 (Orafti). Alternatively, scFOS can be prepared by the enzymatic transfructosylation by sucrase, coupling fructose to sucrose, such as ActiLight (Meiji). scFOS can also be referred to as oligofructose or oligofructan.

The nutritional composition also comprises long-chain fructo-oligosaccharides (lcFOS). In the context of the present invention, lcFOS has an average degree of polymerisation (DP) equal to or above 10, typically in the range of 10-100. Preferably lcFOS has an average DP in the range of 15-50, most preferably lcFOS has an average DP above 20. Fructose units are coupled to each other by beta1,2 linkages. At the terminal end a glucose may or may not be present. Typically, lcFOS is produced from inulin, from which the molecules with a shorter length are removed. A particular type of lcFOS is inulin. Suitable lcFOS products are for instance commercially available, such as Raftilin HP (Orafti) or FibrulineXL (Cosucra).

Preferably the scFOS to lcFOS wt/wt ratio is the range of 9:0.2 to 9:2, more preferably 9:0.5 to 9:1.5.

The NDO in the nutritional composition according to the invention further comprise at least one type of human milk oligosaccharide (HMO) that is 2′-fucosyllactose (2′-FL). scFOS and lcFOS are not considered to be HMOs. In a preferred embodiment the HMOs comprise 90-100 wt % 2′-FL, even more preferably 95 wt %-100 wt % 2′-FL based on total weight of HMOs. In a more preferred embodiment 2′-FL is the only HMO present.

2′-FL may be produced by biotechnological means using specific fucosyltransferases and/or fucosidases either through the use of enzyme-based fermentation technology (recombinant or natural enzymes) or microbial fermentation technology known in the art. In the latter case, microbes may either express their natural enzymes and substrates or may be engineered to produce respective substrates and enzymes. Single microbial cultures and/or mixed cultures may be used. Alternatively, fucosylated oligosaccharides may be produced by chemical synthesis, e.g. from lactose and fucose. The 2′-FL used in the present nutritional composition is not isolated form milk, as ingredients derived from milk are not desired. 2′-FL, not isolated from milk, are also available for example from, Glycom DSM, Denmark or Chr. Hansen, Denmark.

FOS is the sum of scFOS and lcFOS. The wt/wt ratio of FOS to 2′-FL is 9:0.2 to 9:2, preferably 9:0.4 to 9:1. It was found that when ratios were used that were lower, the synergistic effect on SCFA production was no longer observed. With ratios that are higher it is expected that the synergistic effects are also no longer observed.

The nutritional composition preferably comprises 0.10 to 3.60 g NDO that comprise scFOS, lcFOS and 2′-FL per 100 kcal, more preferably 0.15 to 2.85 g per 100 kcal, even more preferably 0.30 to 2.25 g per 100 kcal, even more preferably 0.45 to 1.95 g NDO that comprise scFOS, lcFOS and 2′-FL per 100 kcal. Based on a ready-to-use liquid product the nutritional composition preferably comprises 0.07 to 2.40 g NDO that comprise scFOS, lcFOS and 2′-FL per 100 ml, more preferably 0.10 to 1.90 g per 100 ml, even more preferably 0.20 to 1.50 g per 100 ml, even more preferably 0.30 to 1.30 g NDO that comprise scFOS, lcFOS and 2′-FL per 100 ml. Based on dry weight the nutritional composition preferably comprises 0.5 to 18 wt % NDO that comprise scFOS, lcFOS and 2′-FL, more preferably 0.75 to 14.25 wt %, even more preferably 1.50 to 11.25 wt %, even more preferably 2.25 to 9.75 wt % NDO that comprise scFOS, lcFOS and 2′-FL.

The nutritional composition preferably comprises 0.08 to 1.60 wt % 2′-FL based on dry weight of the nutritional composition, preferably 0.12 to 0.80 wt %. In a preferred embodiment of the invention, 2′-FL is present in an amount of 10 mg to 200 mg per 100 ml ready to drink form, more preferably 15 to 100 mg 2′-FL per 100 ml.

Preferably, short-chain oligosaccharides (such as scFOS and 2′-FL) and long-chain oligosaccharides (such as lcFOS) are present in a weight ratio short-chain to long-chain in the range of 50:50 to 99:1, more preferably 4:1 to 97:3, even more preferably 5:1 to 95:5, even more preferably 7:1 to 95:5, even more preferably 8:1 to 10:1, most preferably about 9:1.

In a preferred embodiment, the wt/wt ratio of FOS (scFOS+lcFOS) to 2′-FL is 9:0.2 to 9:2 and the wt/wt ratio of scFOS to lcFOS is 9:0.2 to 9:2, more preferably the wt/wt ratio of FOS (scFOS+lcFOS) to 2′-FL is 9:0.4 to 9:1 and the wt/wt ratio of scFOS to lcFOS is 9:0.5 to 9:1.5.

In a preferred embodiment according to the present invention the mixture of NDO essentially consists of scFOS, lcFOS and 2′-FL. Preferably the mixture of NDO present in the nutritional composition according to the invention comprises 90 to 100 wt % scFOS, lcFOS and 2′-FL based on weight of total NDO, more preferably 95 to 100 wt %, even more preferably 98 to 100 wt %.

Preferably the present nutritional composition does not comprise galacto-oligosaccharides. This is because galacto-oligosaccharides are typically formed from lactose and contain residual lactose, which is milk derived, and which may not be desired in nutritional compositions for subjects with severe allergy to cow's milk protein or severe lactose intolerance. Also, the present nutritional composition preferably does not comprise 3′-galactosyllactose 3′-GL. Usually 3′-GL is also milk-derived and is therefore preferably not included in nutritional compositions for subjects with severe allergy to cow's milk protein or severe lactose intolerance. Further, the present nutritional composition preferably does not comprise dietary butyrate. Usually dietary butyrate is sourced from cow's milk and is therefore preferably not included in nutritional compositions for subjects with severe allergy to cow's milk protein or severe lactose intolerance.

### Bifidobacteria

Preferably the nutritional composition comprises bifidobacteria, more preferably Bifidobacterium breve. The present nutritional composition preferably contains at least 2.103 colony forming units (cfu) bifidobacteria per gram dry weight of the nutritional composition, more preferably at least 2.104 cfu, even more preferably at least 2.105 cfu bifidobacteria per gram dry weight of the nutritional composition. The present nutritional composition preferably contains 2.103 to 2.1013 colony forming units (cfu) bifidobacteria per gram dry weight of the nutritional composition, preferably 2.104 to 2.1012, more preferably 2.105 to 2.1010, most preferably 2.106 to 2.109 cfu bifidobacteria per gram dry weight of the nutritional composition.

Preferably the Bifidobacterium is a strain of Bifdobacterium breve. Bifidobacterium breve is a Gram-positive, anaerobic, branched rod-shaped bacterium. The B. breve according to the present invention preferably has at least 95% identity of the 16 S rRNA sequence when compared to the type strain of B. breve ATCC 15700, more preferably at least 97% identity (Stackebrandt & Goebel, 1994, Int. J. Syst. Bacteriol. 44:846-849).

Preferred B. breve strains are those isolated from the faeces of healthy human milk-fed infants. Typically these are commercially available from producers of lactic acid bacteria, but they can also be directly isolated from faeces, identified, characterized and produced. Typically B. breve strains are not able to grow on 2′-FL, but can metabolize the building blocks of 2′-FL, fucose and lactose.

Suitable B. breve strains are available. Examples of suitable B. breve strains are BbC50. B. breve C50 was deposited under deposit number CNCM I-2219, under the Budapest Treaty at the Collection Nationale de Cultures de Microorganism, at Institut Pasteur, 25 Rue du Dr Roux, Paris, France on 31 May 1999 by Compagnie Gervais Danone. This strain was published in WO 2001/001785 and in U.S. Pat. No. 7,410,653. This strain is known to have immune stimulating activity and is able to improve the microbiota by reducing pathogens like clostridium, in particular Clostridium perfringens, and Bacteroides fragilis. Furthermore, the strain can produce factors that downregulate intestinal inflammation (Heuvelin et al 2009, Plos one, 4: e5184). These are features that are especially beneficial under conditions when the intestinal microbiota is in disbalance. Another preferred Bifidobacterium breve to use is Bifidobacterium breve CNCM I-5177. B. breve CNCM I-5177 was deposited under the Budapest Treaty at the Collection Nationale de Cultures de Microorganism, at Institut Pasteur, 25 Rue du Dr Roux, Paris, France on 9 Mar. 2017 by Compagnie Gervais Danone. Especially preferred is the B. breve M-16V strain from Morinaga (LMG 23729). This strain, in the presence of non-digestible oligosaccharides including scFOS/lcFOS has demonstrated to improve the microbiota beyond the level of bifidobacteria only (Wopereis et al, 2019, Clin & Transl Allerg. 9:27).

### Protein Component

The nutritional composition comprises a protein component. The terms “protein” and “protein component” encompass intact proteins, peptides, free amino acids and hydrolysed proteins. Hydrolysed proteins may be partially hydrolysed or extensively hydrolysed. The protein component may comprise intact proteins, hydrolysed proteins, peptides and/or free amino acids. The amount of protein component in the nutritional composition is determined by the sum of intact protein, hydrolysed protein, peptides and free amino acids.

Preferably the nutritional composition comprises 1.2 g to 3.6 g protein component per 100 kcal, more preferably 1.6 g to 3.2 g protein component per 100 kcal, even more preferably 2.0 to 2.8 g per 100 kcal. Based on a ready-to-use liquid product the nutritional composition preferably comprises 0.8 to 2.4 g per 100 ml, more preferably 1.1 g to 2.1 g per 100 ml, even more preferably 1.3 to 1.9 g per 100 ml of the protein component. Based on dry weight the nutritional composition preferably comprises 6 to 18 wt %, more preferably 8 to 16 wt %, even more preferably 10 to 14 wt % protein component. Worded alternatively, when the nutritional composition is in a powder form, the protein component is preferably present in an amount of 6 g to 18 g per 100 g dry weight, more preferably 8 to 16 g, even more preferably to 14 g per 100 g dry weight of the composition. The source of the protein component is preferably selected in such a way that the minimum requirements for essential amino acid content are met and satisfactory growth is ensured. Products containing free amino acids that meet these requirements are available, for example Neocate or EleCare or Nutramigen Puramino.

It is preferred that the protein component is non-allergenic or hypoallergenic. Preferably the protein source is not derived from milk proteins. Preferably the protein component is in the form of intact plant protein, hydrolyzed plant protein, and/or free amino acids. Such protein components are suitable for infants or young children that are at risk of or have an allergy for cow's milk protein. Such protein components are suitable for infants and young children that have a severe allergy for cow's milk protein. Plant protein, intact or hydrolyzed, has the advantage that they are suitable for vegans. Suitable plant proteins are rice protein, soy protein or pea protein, and combinations thereof. Rice protein hydrolysates and infant formulas containing such hydrolysates are commercially available.

Thus, in a preferred embodiment of the nutritional composition according to the invention, the protein component is at least one selected from the group consisting of free amino acids, hydrolyzed plant protein and intact plant protein, preferably at least one selected from the group consisting of free amino acids and hydrolyzed plant protein. In a preferred embodiment of the nutritional composition according to the invention, the protein component comprises free amino acids or plant protein as sole protein source, in a preferred embodiment the nutritional composition according to the invention comprises free amino acids as sole protein source.

### Lipid Component

The nutritional composition of the present invention comprises a lipid component. The lipid component is preferably present in an amount of 3 g to 7 g per 100 kcal, more preferably in an amount of 4 g to 6 g lipid per 100 kcal and most preferably in an amount of 4.5 g to 5.5 g lipid per 100 kcal. When in liquid form, e.g. as a ready-to-use liquid, the nutritional composition preferably comprises 2.0 g to 4.7 g lipid per 100 ml, more preferably 2.7 to 4.0 g, even more preferably 3.0 to 3.7 g per 100 ml. Based on dry weight the nutritional composition preferably comprises 15 to 35 wt %, more preferably 20 to 30 wt %, even more preferably 22 to 28 wt % lipid. Worded alternatively, when the nutritional composition is in powder form, the lipids are preferably present in an amount of 15 g to 35 g/100 g dry weight, more preferably 20 to 30 g, even more preferably 22 to 28 g per 100 g dry weight of the composition.

The lipid component comprises vegetable lipid. The presence of vegetable lipid advantageously enables an optimal fatty acid profile high in polyunsaturated fatty acids, such as essential linoleic acid and alpha-linolenic acid, and is more reminiscent to human milk fat. Preferably the nutritional composition comprises at least one, more preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, soy oil, palm oil and palm kernel oil.

Additionally, egg oil and/or fish oil and/or microbial oils such as oil from fungi and algae may be present. Such oils are suitable sources of long-chain polyunsaturated fatty acids such as docosahexaenoic acid (DHA), arachidonic acid (ARA) and/or eicosapentaenoic acid (EPA). Preferably the nutritional composition comprises n3 LC-PUFA, such as EPA and/or DHA, more preferably DHA. Preferably the nutritional composition comprises at least 0.05 wt %, preferably at least 0.1 wt %, more preferably at least 0.2 wt %, DHA based on total fatty acids. Preferably the nutritional composition comprises not more than 2.0 wt %, preferably not more than 1.0 wt % DHA based on total fatty acids. The nutritional composition preferably comprises ARA. Preferably the composition comprises EPA. Preferably the composition comprises EPA, ARA and DHA as such mixtures were found to improve the intestinal barrier function, which is desired for allergic infants and young children. Preferably the ratio EPA to DHA is greater than 0.1 and smaller than 0.3. Preferably the composition comprises DHA:EPA:ARA in a ratio of 1:(0.1-0.2):(0.5-1.0). Preferably the LC-PUFA oils are microbial oils. Fish oils may be more allergenic and less suitable for subjects with multiple food allergies and/or not suitable when a vegan formula is desired.

Preferably the composition does not comprise milk fat. It is not desired to have ingredients derived from milk fat for infants or young children that have severe allergy against cow's milk protein.

### Digestible Carbohydrate Component

The nutritional composition of the present invention comprises a digestible carbohydrate component. Preferably the digestible carbohydrate is maltodextrin and/or glycose syrup. Maltodextrin and glucose syrup are glucose polymers, which are produced from vegetable starch such as for example corn starch or wheat starch by partial hydrolysis. Maltodextrin and glycose syrup are moderately sweet or almost flavorless. Maltodextrin consists of D-glucose units connected in chains of variable length. The glucose units are primarily linked with α(1→4) glycosidic bonds. Maltodextrins are classified by their DE (dextrose equivalent) and have a DE in the range of 3 to 20. The higher the DE value, the shorter the glucose chains and the higher the sweetness. When the DE is higher than 20, the composition is referred to as glucose syrup. Glucose syrup with a high DE is less desired because of the increased sweet taste. In addition, starch, or a maltodextrin with a very low DE, is not preferred as this will thicken the infant formula. Hence the maltodextrin and glucose syrup in the mixture of free galactose and at least one of maltodextrin and glucose syrup have a DE in the range of 3 to 32, preferably in the range of 10 to 25, more preferably in the range of 15 to 25, even more preferably in the range of 17 to 22.

Based on calories the nutritional composition preferably comprises 6 g to 20 g of the digestible carbohydrate component per 100 kcal, more preferably 10 g to 15 g per 100 kcal. When in liquid form, e.g. as a ready-to-use liquid, the nutritional composition preferably comprises 4 g to 13 g digestible carbohydrate component per 100 ml, more preferably 7 g to 10 g per 100 ml. Based on dry weight the nutritional composition preferably comprises 30 wt % to 85 wt %, more preferably 40 wt % to 65 wt % digestible carbohydrates. Worded alternatively, when the nutritional composition is in powder form, the digestible carbohydrates are preferably present in an amount of 40 g to 85 g per 100 g dry weight, more preferably 40 g to 65 g per 100 g dry weight.

Lactose is preferably present in the nutritional composition in an amount of less than 1 wt % and most preferably less than 0.1 wt %, all based on total weight of the digestible carbohydrate component. In another preferred embodiment the composition comprises less than 0.1 wt. % lactose, preferably less than 0.05 wt %, based on dry weight of the nutritional composition. Preferably the lactose content in the nutritional composition is less than 10 mg per 100 kcal. Such formulas are suitable for lactose intolerant subjects. Infants or young children that are severely allergic may have an inflammation in the small intestine, impairing the ability to digest lactose. Furthermore lactose is derived form milk and hence may contain traces of milk protein.

It is not desired to have the complete digestible carbohydrate component existing essentially of glucose. This would result in an amount of free monomeric sugars that is too high, which is not desired but also not allowed for infant and follow on formulas. It would have a taste that is too sweet, sweeter than human or cow's milk. Monomeric carbohydrates also will increase the osmolarity of a nutritional composition and this may not be desired if the nutritional composition has small peptides or free amino acids as protein source instead of intact proteins. In addition, because free glucose is a humectant a powder product like infant formulas, a powder low in free glucose has superior properties. The amount of free glucose is at most 10 wt % based on total weight of the digestible carbohydrate component.

### Nutritional Composition

The nutritional composition according to the invention is not native cow's milk or native milk from another mammal, thus also is not human milk. The present nutritional composition comprises a protein component, a lipid component and a digestible carbohydrate component, wherein the protein component preferably provides 5 to 14%, more preferably 6 to 13%, even more preferably 8 to 11% of the total calories, the lipid component preferably provides 27 to 63%, preferably 36 to 54%, even more preferably 41 to 50% of the total calories, and the digestible carbohydrate component preferably provides 24 to 80%, more preferably 40 to 60% of the total calories.

The composition comprises non-digestible oligosaccharides. Non-digestible oligosaccharides have a caloric density of 2 kcal per g and in the context of the present invention preferably make up 0.2 to 7.2% of total calories.

The nutritional composition preferably comprises 1.2 g to 3.6 g protein component per 100 kcal, more preferably 1.6 g to 3.2 g protein component per 100 kcal, more preferably 2.0 g to 2.8 g protein component per 100 kcal and nutritional composition preferably comprises 3 g to 7 g lipid component per 100 kcal, preferably 4 g to 6 g lipid component per 100 kcal, more preferably 4.5 g to 5.5 g lipid component per 100 kcal and the nutritional composition preferably comprises 6 g to 20 g digestible carbohydrate component per 100 kcal, more preferably 10 g to 15 g digestible carbohydrate component per 100 kcal.

Preferably the nutritional composition has an energy density of 45 to 75 kcal per 100 ml, more preferably 60 to 70 kcal per 100 ml, even more preferably 65 to 70 kcal per 100 ml, when in a ready-to-use form. This density ensures an optimal balance between hydration and caloric intake.

The nutritional composition is preferably in the form of a solid product, preferably in the form of a powder. Suitably, the nutritional composition is in a powdered form, which can be reconstituted with water, to form a ready-to-use liquid. Alternatively, the nutritional composition may be a ready-to-use liquid. The nutritional composition may also be in the form of a liquid concentrate that should be diluted with water in order to obtain a ready-to-use liquid.

The nutritional composition according to the invention preferably is a starter formula, a follow-on formula or a young child formula. This means that the composition that is to be administered is not human milk. Starter formula or follow-on formula or young child formula means that it concerns a composition that is artificially made or in other words that it is a synthetic composition. In the context of the present invention, young child formula can also be named growing-up milk. The present nutritional composition preferably is intended for, or is used for, providing nutrition to an infant or young child.

Starter formulas are intended for young infants from birth to about 4 to 6 months of age and are intended as a substitute for human milk. Typically, starter formulas are suitable to be used as sole source of nutrition. In the context of the present invention this is referred to as a nutritional composition, or starter formula, for the first 6 months of life. A formulas or starter formula can also be suitable for infants 0-12 months. Such formulas are especially known for use for allergic infants. In the context of the present invention such formulas are defined as infant formulas.

Follow-on formulas are intended for infants starting with 4 to 6 months of age to 12 months of age and are intended to be supplementary feedings for infants that start weaning on other foods. In the context of the present invention this is referred to as a nutritional composition, or follow-on formula, for the age of 6 to 12 months.

Young child formula refers to nutritional compositions, artificially made, intended for children of 12 months to up to 48 months of age, in other words for children of 1 to 3 years of age which are intended to be supplementary feedings. In the context of the present invention this is referred to as a nutritional composition, or young child formula, for the age of 12 months up to 48 months, in other words for the age of 1 to 3 years.

Starter formulas and follow-on formulas are subject to strict regulations, for example for the EU regulations no. 609/2013 and no. 2016/127 and Codex Alimentarius for Infant Formula, CODEX STAN 72-1981. Young child formulas preferably follow the directive for follow-on formula. Infant formulas for allergic infants preferably follow the EU directive for food for special medical purposes, FSMP, 2016/128.

Preferably the nutritional composition, more preferably the infant formula, starter formula, follow on formula or young child formula, is free of animal products, so is free of animal proteins such as cow's milk proteins or other cow's milk derived products such as milk fat or lactose. In other words, in a preferred embodiment of the present invention, the nutritional composition according to the invention does not contain animal-derived ingredients.

Preferably further ingredients are present in the nutritional composition like vitamins, minerals, trace elements, nucleotides and other micronutrients as known in the art.

### Application

The nutritional composition according to the invention is preferably used for providing nutrition to an infant or young child. An infant is a human child under the age of 12 months. A young infant is defined as an infant of 0 to 6 months of age. A young child is a human child aged from 1 to 3 years of age (12 months up to 48 months), also called a toddler. In a second aspect, the invention thus relates to the nutritional composition according to the invention, for use in providing nutrition to an infant or young child, preferably an infant of 0 to 12 months of age or a young child aged from 1 to 3 years of age. Preferably the nutritional composition of the present invention is provided to an infant,

Preferably the nutritional composition of the present invention is for use in providing nutrition to an infant or young child that suffers from cow's milk allergy or multiple food allergies, preferably cow's milk protein allergy. Preferably the nutritional composition of the present invention is for use in the dietary management of allergy of an infant or young child that suffers from cow's milk allergy or multiple food allergies, preferably cow's milk protein allergy. Preferably the nutritional composition of the present invention is for use in treating or preventing allergic reaction, preferably allergic reaction to cow's milk protein, in a subject, preferably an infant or young child that suffers from multiple food allergies or cow's milk protein allergy, preferably cow's milk protein allergy. In these cases, the protein component is preferably (hydrolyzed) plant protein, and/or free amino acids, more preferably hydrolyzed plant protein and/or free amino acids. Subjects suffering from cow's milk allergy, in particular severe cow's milk allergy, especially benefit from the nutritional composition with scFOS/lcFOS/2′-FL as this gives an improved fermentation and bifidogenic effect in the intestinal microbiota, which is more comparable to the fermentation obtained with scGOS/lcFOS, whereas the scGOS/lcFOS itself cannot be used in the nutritional composition as scGOS is derived from milk ingredients.

Preferably the nutritional composition of the present invention is for use in providing nutrition to an infant or young child that is lactose intolerant. In that case the amount of lactose should be low, below 1 wt % based on total digestible carbohydrates, more preferably below 0.1 wt %. In this embodiment it is preferred that the amount of lactose is below 0.1 wt %, preferably below 0.05 wt %, based on dry weight of the nutritional composition. Preferably the lactose content in the nutritional composition is lower than 10 mg per 100 kcal in this embodiment. Such nutritional compositions are suitable for lactose intolerant subjects. Infants or young children that are severely allergic may have an inflammation in the small intestine, impairing the ability to digest lactose. Furthermore lactose coming from milk may have traces of milk protein.

The inventors found that fermentation, by microbiota of breastfed infants, of scFOS/lcFOS when compared with scGOS/lcFOS resulted in lower amounts of total short-chain fatty acids, acetic acid and lactic acid, and higher amounts of propionic acid. Surprisingly a mixture of scFOS/lcFOS/2′-FL in a ratio of about 9:1:1 resulted in a higher amount of total short-chain fatty acids, a higher amount of acetic acid and a higher amount of lactic acid and a lower amount of propionic acid. The metabolic profile was closer to that observed with scGOS/lcFOS fermentation and as can be expected for infants receiving breast milk. This effect was synergistic, as it could not be expected based on the results with 2′-FL or scFOS/lcFOS alone. Additionally, the amount of gas formed was lower than expected. When scFOS/lcFOS/2′-FL was tested in a weight ratio of 0.9:0.1:1, the synergistic effect was not observed.

In a subsequent experiment with faecal material from breastfed infants and from toddlers similar results were obtained. The mixture of scFOS/lcFOS/2′-FL performed better than expected and resulted in an age-appropriate metabolic profile that is associated with less risk of atopic disease and also resulted in higher levels of bifidobacteria compared to a composition that did not comprise all three components scFOS, lcFOS and 2′-FL of the mixture of scFOS/lcFOS/2′-FL. Even better results were observed with a mixture of scFOS/lcFOS/2′-FL that further contained Bifidobacterium breve. This composition performed better than scFOS/lcFOS with B. breve or scFOS/lcFOS/2′-FL without B. breve.

Hence, preferably the nutritional composition comprising scFOS/lcFOS/2′-FL, preferably further comprising B. breve, of the present invention is for use in increasing the amount of organic acids formed by the intestinal microbiota upon fermentation of the mixture of non-digestible oligosaccharides. Preferably the nutritional composition comprising scFOS/lcFOS/2′-FL, preferably further comprising B. breve, of the present invention is for use in increasing the amount of bifidobacteria in the intestine. Preferably the nutritional composition comprising scFOS/lcFOS/2′-FL, preferably further comprising B. breve, of the present invention is for use in increasing the relative amount of L-lactic acid and decreasing the relative amount of propionic acid relative to the total amount of organic acids formed by the intestinal microbiota.

In the context of the present invention, increasing or decreasing is compared to when a nutritional composition not comprising all three components of the mixture of scFOS, lcFOS and 2′-FL and not comprising galacto-oligosaccharides is consumed.

The invention also concerns a method for improving intestinal health in a human subject compared to the intestinal health in a human subject by improving the intestinal microbiota metabolic profile and/or level of intestinal microbiota activity, and/or increasing intestinal bifidobacteria in a human subject when compared to a human subject fed exclusively or predominantly a nutritional composition without the combination of scFOS, lcFOS and 2′-FL and without galacto-oligosaccharides, comprising administering the nutritional composition according to the invention to the human subject. In other words, the invention concerns the present nutritional composition for use in improving intestinal health in a human subject compared to the intestinal health in a human subject by improving the intestinal microbiota metabolic profile and/or level of intestinal microbiota activity, and/or increasing intestinal bifidobacteria in a human subject when compared to a human subject fed exclusively or predominantly a nutritional composition without the combination of scFOS, lcFOS and 2′-FL and without galacto-oligosaccharides.

The invention can also be worded as non-digestible oligosaccharides for the manufacture of a nutritional composition for improving intestinal health in a human subject compared to the intestinal health in a human subject by improving the intestinal microbiota metabolic profile and/or level of intestinal microbiota activity, and/or increasing intestinal bifidobacteria in a human subject when compared to a human subject fed exclusively or predominantly a nutritional composition without the combination of scFOS, lcFOS and 2′-FL and without galacto-oligosaccharides, wherein the non-digestible oligosaccharides comprise a mixture of short-chain fructo-oligosaccharides (scFOS), long-chain fructo-oligosaccharides (lcFOS) and 2′-fucosyllactose (2′-FL), wherein the wt/wt ratio of the sum of scFOS and lcFOS to 2′-FL is 9:0.2 to 9:2, preferably 9:0.4 to 9:1.

Preferably the nutritional composition comprising scFOS/lcFOS/2′-FL, preferably further comprising B. breve, is for use in increasing the relative amount of L-lactic acid and for decreasing the relative amount of propionic acid and branched short-chain fatty acids formed, when compared to the relative amount of L-lactic acid, propionic acid and branched chain fatty acids formed by the microbiota of infants with an age of 0 to 6 months, that are exclusively fed by formula not comprising all three components of the mixture of scFOS, lcFOS and 2′-FL and not comprising galacto-oligosaccharides.

Whereas in young infants that are exclusively breastfed a metabolic profile high in lactic acid and acetic acid is desired, in older infants that are weaned to a more adult diet and young children, a metabolic profile high in lactic acid is less desired. With the infant increasing in age the microbiota develops and lactic acid utilizing bacteria develop which metabolize the lactic acid formed by lactic acid producing bacteria, such as bifidobacteria, to propionic acid and in particular butyric acid. Such a development of the metabolic profile is associated with beneficial effects. Infants with eczema are characterized by decreased levels of both isomers of lactic acid and increased levels of propionic acid and butyric acid at the age of 12 weeks. However, this pattern is reversed at 26 weeks of age, with infants having eczema showing increased levels of lactic acid and decreased levels of propionic acid and butyric acid (Wopereis et al Journal of Allergy and Clinical Immunology, 2018, 141 (4) 1334-1342.e5). Especially the presence of butyric acid is thought to have a beneficial effect.

Hence, preferably the nutritional composition comprising scFOS/lcFOS/2′-FL, preferably further comprising B. breve, upon fermentation of the mixture of non-digestible oligosaccharides, the amount of butyric acid formed is relatively increased, fueled by a continuous formation of L-lactic acid, and the amount of branched short-chain fatty acids and ammonia is relatively decreased when compared to the total amount of organic acids formed by the microbiota of infants or young children.

### EXAMPLES

### Example 1: In Vitro Fermentation of scFOS/lcFOS/2′-FL by Microbiota of Breastfed Infant Shows an Organic Acid Production in Profile Closer to the Organic Acids Produced Upon GOS/lcFOS Fermentation

Faecal samples were collected from a formula fed infant (4 months of age) and from two breastfed infants (3 and 4 months of age). The infants were without gastrointestinal problems and did not use antibiotics in the last month. Faecal samples were pooled, homogenized, divided in smaller aliquots, and mixed with glycerol (10%) in an anaerobic cabinet. Subsequent aliquot storage was at −80° C.

The non-digestible oligosaccharides were added at a concentration of 200 mg non-digestible oligosaccharides (DP>2) per 6 ml of faeces suspension. Source of GOS was Vivinal®GOS (Friesland Campina), source of scFOS was Raftilose P95® (Orafti), source of lcFOS was Raftilin HP® (Orafti), and the source of 2′-FL was Jennewein (now part of Chr Hansen). For Vivinal®GOS also lactose was taken as a non-digestible as there is no digestion step.

For the experiment, the faecal pool was defrosted in a water bath for 20 minutes at 37° C. The faecal pool was put thereafter in the anaerobic cabinet. Faeces was mixed with fermentation medium as 1:5 in a falcon tube. Samples of this faecal suspension were taken at t=0 and 6 ml of this suspension was added to a sterile falcon tube together with substrate of interest and mixed thoroughly. Next, 6 ml of the faeces/substrate suspension was put in a dialysis tube and air was removed in the empty space. The dialysis tube was put in a 100 ml Scott bottle filled with 100 ml dialysis medium. The Scott bottles were closed and incubated at 37° C. Samples of the dialysis medium (dialysate) and faecal suspension (lumen) were taken at t=24 and t=48 hours for determination of SCFA, L-lactic acid, and gas volume. Fermentation medium (McBain and Macfarlane) contained buffered peptone water 3.0 g/l, Yeast Extract 2.5 g/l, Tryptone 3.0 g/l, L-Cysteine-HCl 0.4 g/l, Bile salts 0.05 g/l, K2HPO4.3H2O 2.6 g/l, NaHCO₃0.2 g/l, NaCl 4.5 g/l, MgSO4.7H2O 0.5 g/l, CaCl2. 2H2O 0.3 g/l, FeSO4.7H2O 0.005 g/l. Ingredients were added one by one in 800 ml water, pH was adjusted to 5.5±0.1 with K2HPO4 or NaHCO₃ and volume was filled up to 1 litre. Medium was sterilized for 15 minutes at 121° C. and put in the anaerobic cabinet at least 16 hours before use.

Dialysis medium contained K2HPO4.3H2O 2.6 g/l, NaHCO₃0.2 g/l, NaCl 4.5 g/l, MgSO4.7H2O 0.5 g/l, CaCl2. 2H2O 0.3 g/l, FeSO4.7H2O 0,005 g/l. pH was adjusted to 5.5±0.1 with K2HPO4 or NaHCO₃. Medium was not sterilized because of forming of sediment. The medium was put in the anaerobic cabinet at least 16 hours before use.

Gas volume was determined with a unit to measure pressure and volume. The bottles were shaken thoroughly before measuring.

The SCFA, acetic, propionic, n-butyric, iso-butyric, n-valeric, and isovaleric acids were quantitatively determined using a Shimadzu-GC2025 gas chromatograph with a flame ionization detector. As mobile phase hydrogen was used. The levels of SCFA were determined using 2-ethylbutyric acid as an internal standard. From the peak area a calibration curve was constructed and the concentration in the samples was calculated.

Lactic acid was determined enzymatically using an L-lactic acid detection kit with L-lactate dehydrogenase (Boehringer Mannheim, Mannheim, Germany). First, samples were centrifuged for 10 min at 13.000 rpm at 4° C., then the supernatant was heated for 10 min at 100° C. to inactivate all enzymes and then the samples were centrifuged for 10 minutes at 13.000 rpm.

### Results:

Results are shown in Table 1.

TABLE 1
Amounts or organic acid and gas formed over time per g NDO upon
fermentation of non-digestible oligosaccharides by infant faecal samples.

| NDO tested | t = 24 h | t = 48 h |
|---|---|---|
| | Acetic acid (μmol/g NDO) | |
| 2′-FL | 6450 | 7050 |
| ScGOS/lcFOS | 6109 | 7034 |
| scFOS/IcFOS | 4975 | 5475 |
| scFOS/lcFOS/2′-FL 8.1:0.9:1 | 5050 | 5575 |
| Theoretical | 5123 | 5633 |
| scFOS/lcFOS/2′-FL 0.9:0.1:1 | 5225 | 5600 |
| Theoretical | 5713 | 6263 |
| | Propionic acid (μmol/g NDO) | |
| 2′-FL | 60 | 97.5 |
| scGOS/lcFOS | 46.3 | 61.7 |
| scFOS/IcFOS | 77.5 | 97.5 |
| scFOS/lcFOS/2′-FL 8.1:0.9:1 | 57.5 | 77.5 |
| Theoretical | 75.75 | 97.5 |
| scFOS/lcFOS/2′-FL 0.9:0.1:1 | 65 | 85 |
| Theoretical | 68.75 | 97.5 |
| | L-lactic acid (μmol/g NDO) | |
| 2′-FL | 3345 | 3645 |
| scGOS/IcFOS | 5717 | 7219 |
| scFOS/IcFOS | 3875 | 4175 |
| scFOS/lcFOS/2′-FL 8.1:0.9:1 | 4480 | 4780 |
| Theoretical | 3822 | 4122 |
| scFOS/lcFOS/2′-FL 0.9:0.1:1 | 3300 | 3600 |
| Theoretical | 3610 | 3910 |
| 2′-FL | Gas (ml/g NDO) | |
| | 23.5 | 35 |
| scGOS/lcFOS | 18.5 | 26.5 |
| scFOS/IcFOS | 24 | 34.5 |
| scFOS/lcFOS/2′-FL 8.1:0.9:1 | 21 | 31 |
| Theoretical | 24 | 34.6 |
| scFOS/lcFOS/2′-FL 0.9:0.1:1 | 25 | 35.5 |
| Theoretical | 23.8 | 34.8 |
| | Total SCFA (μmol/g NDO) | |
| 2′-FL | 6530 | 7177.5 |
| scGOS/lcFOS | 6171 | 7119 |
| scFOS/IcFOS | 5072.5 | 5602.5 |
| scFOS/lcFOS/2′-FL 8.1:0.9:1 | 5127.5 | 5682.6 |
| Theoretical | 5218.3 | 5760 |
| scFOS/lcFOS/2′-FL 0.9:0.1:1 | 5310 | 5715 |
| Theoretical | 5801.3 | 6390 |

For all NDO tested almost no butyric acid was found.

As expected fermentation of GOS/lcFOS by faecal material of a young infant that is fully breastfed resulted in a high amount of acetic acid, a low amount of propionic acid, and a high amount of L-lactic acid at t=24 h and t=48 h.

2′-FL alone also resulted in a high amount of acetic acid, and a slightly lower amount of L-lactic acid and a slightly higher amount of propionic acid when compared to GOS/lcFOS.

Fermentation of scFOS/lcFOS when compared to GOS/lcFOS resulted in a lower amount of acetic acid, a lower amount of SCFA, a higher amount of propionic acid and a lower amount of L-lactic acid, and also the total amount of SCFA and total organic acids (being the sum of SCFA and lactic acid) formed was lower.

A mixture of scFOS/lcFOS/2′-FL 8.1:0.9:1 surprisingly showed the formation of a higher amount of L-lactic acid and a lower amount of propionic acid than expected based on 2′-FL and scFOS/lcFOS alone. The metabolic profile thus was closer to that observed with the GOS/lcFOS fermentation and closer to what can be expected in the large intestine of an exclusively human milk fed young infant. The total amount of organic acids formed was higher than expected and closer to that of the GOS/lcFOS fermentation. Additionally the amount of gas formed was a bit lower than expected.

However, when scFOS/lcFOS/2′-FL was tested in a 0.9:0.1:1 ratio the synergistic effects were not observed. On the contrary, the amounts of acetic acid and L-lactic acid were lower than expected. In addition the amount of gas formed was not lower.

These results are indicative of a beneficial effect of a nutritional composition comprising scFOS, lcFOS and 2′-FL, wherein the wt/wt ratio of the sum of scFOS and lcFOS to 2′-FL is 9:0.2 to 9:2 on the microbiota activity of infants.

### Example 2: Effect of 2′-FL and scFOS/lcFOS on Bifidobacteria Formation after Fermentation of Microbiota of Breastfed Infant and the Effect of Added Bifidobacterium breve

Faecal samples were obtained from a breast fed infant of 4 months of age and stored at −80° C. until use.

### Probiotic Strain

Frozen pellets (counted stock) of Bifidobacterium breve M-16V (Morinaga) containing a pNZ44St plasmid conferring Streptomycin resistance. This enables to follow strain by selective plating on TOS-propionate agar with streptomycin supplement.

Per feeding the final dose of B. breve was 1×10⁸ cfu per well (1.6 ml).

The following experimental arms #1-#6 were tested:

| | |
|---|---|
| #1 | B. breve control |
| #2 | 2′-FL − 5 g/L |
| #3 | scFOS/lcFOS 9:1 − 5 g/l |
| #4 | scFOS/lcFOS/2′-FL 9:1:1.25 − 5.625 g/l |
| #5 | scFOS/IcFOS 9:1 − 5 g/l + B. breve |
| #6 | scFOS/lcFOS/2′-FL 9:1:1.25 − 5.625 g/l + B. breve |

Colonic Microbiota Medium for faecal slurry fermentations: Yeast extract 1 g/L, Ammonium sulphate 2 g/L, K₂HPO₄ 2 g/L, NaHCO₃3.2 g/L, NaCl 4.5 g/L, MgSO₄·7H₂O 0.5 g/L, CysteinHCl 0.5 g/L, CaCl₂·2H₂O 0.4 g/L, Bile Salts 25 mg/L (Sigma), 2 ml metal solution ((containing per L: 500 mg EDTA, 200 mg FeSO₄·7H₂O, 10 mg ZnSO₄·7H₂O, 3 mg MnCl₂·7H₂O, 30 mg H₃BO₃, 20 mg CoCl₂·6H₂O, 1 mg CuCl₂·2H₂O, 2 mg NiCl₂·6H₂O, 3 mg NaMoO₄·2H₂O, 7.5 mg NaSeO₃), and vitamin solution (containing per L: 1 g menadione, 2 g biotin, 2 g pantothenate, 10 g nicotinamide, 0.5 g cobalamine, 4 g thiamine, 5 g p-aminobenzioc acid; filter-sterilised), haemin (10 mg/L), Porcine stomach Mucus 2.5 g/L, and 25 mg/L bile acids (Sigma).

Faecal samples were thawed under anaerobic conditions and approximately a 4% (w/v) suspension of the faecal samples were made in age adapted Colonic Microbiota medium containing 25 mM acetic acid and 12 mM lactic acid, 1 g/l tryptone, without carbon source, adjusted to pH 5.5 (to mimic the pH of breast fed infant faces). The diluted faecal samples were homogenized, allowed to sediment for 5 minutes, then filtered over a tea sieve to remove large particles and subsequently filtered over a Millex 100 μm vacuum filter. After initial faecal community stabilization (the first 4 hours of fermentation), medium was used without adding selective acetic acid and lactic acid.

A Biolector Pro plate (BOH2 round well, M2P-labs) with pH optodes was used. Respectively 6 wells of this plate were filled with 1.52 ml of the faecal solutions. One feeding row of the plate was filled with sterile 3M NaOH. The wells of the other feeding row for the night feedings were filled with the following solutions, respectively: #1. B. breve control (1% glucose stock solution); #2. 2′-FL (10% stock solution); #3, and #5. scFOS/lcFOS (10% stock solution); #4, and #6. scFOS/lcFOS/2′-FL (11.25% stock solution).

After this, plates were sealed with ventilated silicone foil with slits. The plate was incubated (85% moisture, 37° C., 600 rpm, anaerobic (90% N2, 5% CO2, 5% H₂)) in BioLector Pro. At T=0, respectively, 80 microliters of the sterile carbohydrate stock solutions 1 till 6, order as described above, were added as bolus feeding. To #1, #5 and #6 thawed pellet of B. breve M-16V was added in a concentration of 1×10⁸ cfu/well. The experiment was started with pH 5.5 with continuous pH control. During day time 2 times manual feeding every 4 hours was supplied followed by automatic continuous slow carbohydrate feeding (80 μl overnight) via the BioLector system via the microfluidic system.

At 28 h samples were taken for total bifidobacteria colony forming unit count by spot-plating method on TOS-MUP agar and for total B. breve M-16V specific colony forming unit count on TOS-MUP with streptomycin.

### Results:

The amount of bifidobacteria was increased when a combination of 2′-FL and scFOS/lcFOS was used compared to 2′-FL or scFOS/lcFOS alone. The addition of B. breve further increased the amount of total bifidobacteria.

Similarly the amount of bifidobacteria was increased if B. breve was added to scFOS/lcFOS and the amount was further increased if additional 2′-FL was present. This is indicative for a further improved effect with scFOS/lcFOS/2′-FL+B. breve compared to scFOS/lcFOS+B. breve or scFOS/lcFOS/2′-FL without B. breve. See Table 2.

TABLE 2
Total bifidobacteria (¹⁰log CFU/ml)

| | | Total bifidobacteria (¹⁰log CFU/ml) |
|---|---|---|
| #1 | B. breve M-16V | 8.0 |
| #2 | 2′-FL | 8.3 |
| #3 | scFOS/lcFOS | 8.3 |
| #4 | scFOS/lcFOS/2′-FL | 8.8 |
| #5 | scFOS/lcFOS + B. breve | 8.8 |
| #6 | scFOS/lcFOS/2′-FL + B. breve | 9.3 |

The increase of the B. breve strain M16-V was also determined, and was highest in the scFOS/lcFOS/2′-FL group. Although B. breve M-16V cannot consume 2′-FL directly, it can consume the 2′-FL building blocks (fucose and lactose) and so this indicates that cross-feeding is present. Also the total increase in total bifidobacteria in the scFOS/lcFOS/2′-FL group is higher than the increase of B. breve M-16V alone and this indicates that also the endogenous bifidobacteria are increased and to the highest extent when scFOS/lcFOS/2′-FL+B. breve M-16V is present.

TABLE 3
B. breve M-16V specific (¹⁰log CFU/ml)

| | | B. breve M-16V (¹⁰log CFU/ml) |
|---|---|---|
| #1 | B. breve M-16V | 8.0 |
| #5 | scFOS/lcFOS + B. breve | 8.8 |
| #6 | scFOS/lcFOS/2′-FL + B. breve | 9.2 |

### Example 3: Effect of 2′-FL and scFOS/lcFOS on Fermentation Profiles in Microbiota of a Toddler and the Effect of Added Bifidobacterium breve

Whereas in young infants that are exclusively breastfed a metabolic profile high in lactic acid and acetic acid is desired, in older infants that are weaned to a more adult diet and toddlers, a metabolic profile high in lactic acid is less desired. With the infant increasing in age the microbiota develops and lactic acid utilizing bacteria develop which metabolize the lactic acid formed by lactic acid producing bacteria such as bifidobacteria to propionic acid and in particular butyric acid. Therefore it was examined which organic acids were formed upon fermentation of the non-digestible oligosaccharides by faecal samples obtained from a toddler.

Faecal samples were obtained from a young child of 1.5 years of age and stored at −80° C. until use. Experiments were performed in a similar way as in example 2. The pH for the age adapted colonic medium was pH 6.0 (to mimic the pH of toddler faeces).

Samples for analysis were obtained as follows. After 4 hours the experiment was paused, the faecal slurry of each well was harvested and centrifuged for a short time, all under aseptic anaerobic conditions.

The supernatant was frozen for further analyses while the faecal pellet was resuspended in fresh medium with carbohydrates and pipetted back in original well of BOH2 plate. The procedure was repeated again for 3 days. During day time 2 times manual feeding every 4 hours was supplied followed by automatic continuous slow carbohydrate feeding (80 μl overnight) via the BioLector system via the microfluidic system.

Faecal supernatants of the faecal fermentations were thawed and aliquoted to quantitatively determine the SCFA acetic, propionic, n-butyric, isovaleric and n-valeric acids by gas chromatography, ammonia concentration by the rapid Ammonia kit (Megazyme) and after heat inactivation D- and L-lactate by the D-lactic acid/L-lactic acid kit (R-Biopharm AG).

### Results:

Base consumption, or acid formation, was slower with 2′-FL when compared to scFOS/lcFOS or scFOS/lcFOS/2′-FL. With scFOS/lcFOS/2′-FL plus added B. breve a higher NaOH consumption was observed when compared to scFOS/lcFOS/2′-FL in the absence of B. breve or when compared to scFOS/lcFOS+B. breve.

The formation of SCFA formed was in accordance with the NaOH consumption. Higher levels were observed for the scFOS/lcFOS/2′-FL combination when compared to scFOS/lcFOS or 2′-FL alone. This was applicable for acetic acid, propionic acid and butyric acid.

Furthermore, a slightly higher level of cumulative total SCFA was observed in scFOS/lcFOS/2′-FL with added B. breve compared to scFOS/lcFOS/2′-FL without B. breve or compared to scFOS/lcFOS with B. breve. This was especially the case for acetic acid and butyric acid, and the amount of propionic acid was relatively lower in the scFOS/lcFOS/2′-FL with added B. breve group. Also levels branched chain fatty acids (BCFA) and ammonia were lowest in the scFOS/lcFOS/2′-FL with B. breve which is indicative for reduced proteolytic activity. See table 4.

TABLE 4
Cumulative SCFA, BCFA and ammonia data over 6 intervals of 4 h during 3 days

| | SCFA (mmol/l) | | | | BCFA (mmol/l) | | | ammonia |
|---|---|---|---|---|---|---|---|---|
| | acetic | propionic | butyric | total | iso-butyric | iso-valeric | total | (mmol/l) |
| | acid | acid | acid | SCFA | acid | acid | BCFA | ammonia |
| 2′-FL | 255 | 48 | 21 | 324 | 5 | 7 | 12 | 97 |
| scFOS/lcFOS | 319 | 56 | 31 | 406 | 6 | 8 | 14 | 109 |
| scFOS/lcFOS/2′-FL | 323 | 56 | 33 | 412 | 5 | 8 | 13 | 96 |
| scFOS/lcFOS + B. breve | 341 | 56 | 30 | 427 | 5 | 8 | 13 | 98 |
| scFOS/lcFOS/2′-FL + | 369 | 49 | 39 | 457 | 4 | 6 | 10 | 91 |
| B. breve | | | | | | | | |

TABLE 5
Lactic acid and butyric acid over time

| | 4 | 8 | 28 | 32 | 52 | 56 | cumu- |
|---|---|---|---|---|---|---|---|
| | h | h | h | h | h | h | lative |
| | L-lactic acid (mmol/l) | | | | | | |
| 2′-FL | 0 | 4 | 0 | 0 | 0 | 0 | 4 |
| scFOS/lcFOS | 0 | 7 | 10 | 0 | 0 | 0 | 17 |
| scFOS/lcFOS/2′-FL | 2 | 8 | 9 | 2 | 0 | 0 | 21 |
| scFOS/lcFOS + B. breve | 0 | 10 | 12 | 4 | 0 | 0 | 26 |
| scFOS/lcFOS/2′-FL + B. breve | 0 | 10 | 9 | 8 | 0 | 0 | 28 |
| | butyric acid (mmol/l) | | | | | | |
| 2′-FL | 0 | 2 | 4 | 5 | 5 | 5 | 21 |
| scFOS/lcFOS | 0 | 5 | 4 | 6 | 9 | 8 | 32 |
| scFOS/lcFOS/2′-FL | 0 | 5 | 4 | 6 | 9 | 8 | 32 |
| scFOS/lcFOS + B. breve | 0 | 3 | 4 | 7 | 8 | 8 | 30 |
| scFOS/lcFOS/2′-FL + B. breve | 0 | 3 | 4 | 7 | 13 | 13 | 40 |

Interestingly, upon fermentation by the microbiota of the young child, the level of L-lactic acid decreased over time. L-Lactic acid was formed initially, but after adaptation, the L-lactic acid levels were decreased. Initially the level of butyric acid was low, but after adaptation more butyric acid was formed. Compared to 2′-FL alone or scFOS/lcFOS alone, the combination scFOS/lcFOS/2′-FL showed a higher cumulative lactic acid production and butyric acid production, with scFOS/lcFOS/2′-FL showing L-lactic acid formation more continuously.

The presence of B. breve prolonged the formation of lactic acid even in a more continuous way, thereby beneficially providing in an extended way a substrate for the lactic acid utilizing bacteria and butyric acid generation. Likewise a more continuous formation with scFOS/lcFOS/2′-FL with B. breve was also observed when compared to scFOS/lcFOS with B. breve.

The highest levels of butyric acid were formed in the presence of scFOS/lcFOS/2′-FL+B. breve, see table 5.

Levels of branched chain fatty acids and ammonia were low, and over time lowest for the scFOS/lcFOS/2′-FL plus B. breve combination, when compared to scFOS/lcFOS+B. breve and scFOS/lcFOS/2′-FL without B. breve which is indicative for less proteolytic fermentation.

Total bifidobacteria was increased when B. breve M-16V was added, so for scFOS/lcFOS/2′-FL+B. breve, when compared to scFOS/lcFOS/2′-FL or scFOS/lcFOS+B. breve (data not shown).

Also here the addition of B. breve M-16V contributed to positive metabolic effects and bifidogenic effect.

The presence of bifidobacteria in toddlers is beneficial as the lactic acid formed by the bifidobacteria is used by lactic acid utilizing bacteria to convert to butyric acid in older infants and young children.

Overall, the results of examples 1, 2 and 3 show that intervention with adding 2′-FL on top of scFOS/lcFOS, with scFOS/lcFOS in a weight ratio of 9:0.2 to 9:2 and sum of scFOS+lcFOS to 2′-FL in a weight ratio of 9:0.2 to 9:2, gives an age appropriate metabolic profile, with high levels of lactic acid and acetic acid and low propionic acid in young, exclusively breastfed infants, and more butyric acid formed at later age stages. This metabolic profile is further improved when a bifidobacteria strain is present, in particular B. breve.

### Example 4

An amino acid based infant formula in powder form, packed with on the package instructions for reconstitution with water and an indication that it is for the use for the dietary management of infants of 0-12 months that suffer from cow's milk allergy, multiple food allergies and other indications where an amino acid based formula is needed.

About 14.4 g of powder is reconstituted with water to 100 ml of formula.

Per 100 g powder to composition comprises:
474 kcal
13.2 g protein equivalent, free amino acids (L-Arg-L-Asp, L-Leu, L-Lys, L-Glu, L-Pro, L-Val, L-Ile, Gly, L-Thr, L-Phe, L-Tyr, L-Ser, L-His, L-Ala, L-Cys, L-Trp, L-Met)
50.2 g digestible carbohydrates (mainly glucose syrup)
23.6 g lipid (mainly vegetable lipid and containing microbial oil delivering arachidonic acid, and docosahexaenoic acid)
4.64 g non-digestible oligosaccharide mixture consisting of
   4.0 scFOS (source Raftilose®P95)
   0.45 g lcFOS (source Raftiline®HP)
   0.14 g 2′-fucosyllactose (Chr Hansen)
B. breve M-16V (Morinaga) 10⁸ cfu per g powder
minerals, trace elements, vitamins and other micronutrients according to international directives for infant formulas

### Example 5

A rice protein hydrolysate infant formula in powder form, packed with on the package instructions for reconstitution with water and an indication that it is for the use for the dietary management of infants of 0-12 months that suffer from cow's milk allergy.

About 13.5 g of powder is reconstituted with water to 100 ml of formula.

Per 100 g powder to composition comprises:
495 kcal
12.4 g protein equivalent, rice protein hydrolysate, L-Trp, L-Tyr, L-Ile
50.6 g digestible carbohydrates (mainly maltodextrin)
26.0 g lipid (mainly vegetable lipid and containing microbial oil delivering arachidonic acid and docosahexaenoic acid
4.6 g non-digestible oligosaccharide mixture consisting of
   4.0 scFOS (source Raftilose®P95)
   0.45 g lcFOS (source Raftiline®HP)
   0.45 g 2′-fucosyllactose (Chr Hansen)
minerals, trace elements, vitamins and other micronutrients according to international directives for infant formulas and FSMP regulation.

### Example 6

An amino acid based formula in powder form, packed with on the package instructions for reconstitution with water and an indication that it is for the use for the dietary management of children from 1 year of age onwards that suffer from severe food allergies and other indications where an amino acid based formula is needed.

About 21.1 g of powder is reconstituted with water to 100 ml of formula.

Per 100 g powder to composition comprises:
472 kcal
14.8 g protein equivalent, free amino acids (L-Ser, L-GIn, Gly, L-Ala, L-Leu, L-Lys, L-Thr, L-Tyr, L-Val, L-Ile, L-Pro, L-Cys, L-His, L-Phe, L-Met, L-Arg, L-Trp)
51.4 g digestible carbohydrates (mainly glucose syrup)
23 g lipid (vegetable lipid)
1.90 g non-digestible oligosaccharide mixture consisting of
   1.63 scFOS (source Raftilose®P95)
   0.18 g lcFOS (source Raftiline®HP)
   0.09 g 2′-fucosyllactose (Chr Hansen)
minerals, trace elements, vitamins and other micronutrients as known in the art

1. A nutritional composition comprising lipids, digestible carbohydrates, a mixture of non-digestible oligosaccharides comprising short-chain fructo-oligosaccharides (scFOS), long-chain fructo-oligosaccharides (lcFOS) and 2′-fucosyllactose (2′-FL), wherein the wt/wt ratio of the sum of scFOS and lcFOS to 2′-FL is 9:0.2 to 9:2, wherein scFOS has an average degree of polymerisation of less than 10, wherein lcFOS has an average degree of polymerisation equal to or above 10, wherein the nutritional composition is not native milk from a mammal.

2. The nutritional composition according to claim 1, wherein the wt/wt ratio of scFOS to lcFOS is 9:0.2 to 9:2.

3. The nutritional composition according to claim 1, which comprises bifidobacteria.

4. The nutritional composition according to claim 1, which comprises less than 1 wt % lactose based on total digestible carbohydrates.

5. The nutritional composition according to claim 1, which does not contain galacto-oligosaccharides.

6. The nutritional composition according to claim 1, which comprises free amino acids or plant protein as sole protein source.

7. The nutritional composition according to claim 1, which is free of ingredients derived from non-human mammalian milk.

8. The nutritional composition according to claim 1, wherein 98-100 wt % of the total weight of non-digestible oligosaccharides are scFOS, lcFOS and 2′-FL.

9. The nutritional composition according to claim 1, which is an infant formula, a follow-on formula or a young child formula.

10. The nutritional composition according to claim 1, wherein the non-digestible oligosaccharides are present in an amount of 0.1 to 1.9 g per 100 ml ready to use liquid, or 0.75 to 14.25 wt % based on dry weight of the composition.

11.-17. (canceled)

18. The nutritional composition according to claim 3, wherein the composition comprises the bifidobacteria Bifidobacterium breve.

19. A method for providing nutrition to a human subject suffering from food allergy, the method comprising feeding the subject the nutritional composition according to claim 1.

20. The method according to claim 19, wherein the food allergy is cow's milk protein allergy.

21. The method according to claim 19, wherein the human subject is an infant or young child.

22. A method for improving intestinal health in a human subject compared to the intestinal health in a human subject by improving the intestinal microbiota metabolic profile and/or level of intestinal microbiota activity, and/or increasing intestinal bifidobacteria in a human subject when compared to a human subject fed exclusively or predominantly a nutritional composition without the combination of scFOS, lcFOS and 2′-FL and without galacto-oligosaccharides, the method comprising administering the nutritional composition of claim 1.

23. The method of claim 22, wherein the human subject is an infant or young child.
